(19) United States
(12) Patent Application Publication
(10) Pub. No.: US 2011/0319871 A1
(43) Pub. Date: Dec. 29, 2011

(54) INFECTIOUS DISEASE CELLULAR IMMUNOTHERAPY

(75) Inventor: Gary W. Wood, Kansas City, MO (US)

(73) Assignee: TVAX BIOMEDICAL, LLC, KANSAS CITY, MO (US)

(21) Appl. No.: 13/254,059

(22) PCT Filed: Mar. 9, 2010
(86) PCT No.: PCT/US10/26635

§ 371 Date: Aug. 31, 2011

Related U.S. Application Data
(60) Provisional application No. 61/209,502, filed on Mar. 9, 2009.

(51) Int. Cl. A61M 37/00

(52) U.S. Cl. 604/522

(57) ABSTRACT

Methods for treating infectious diseases in persons are provided. A person having an infectious disease may be vaccinated with a vaccine designed to induce an immune response against an infectious agent causing the infectious disease. Primed T-lymphocytes are removed from the person and the primed T-lymphocytes are stimulated to differentiate into effector T-lymphocytes in vitro. The effector T-lymphocytes are stimulated to proliferate, in vitro, and the effector T-lymphocytes are infused back into the person.

### CROSS-REFERENCE TO RELATED APPLICATIONS

This application claims priority to PCT Application No. PCT/U.S. 2010/026635 entitled “INFECTIOUS DISEASE CELLULAR IMMUNOTHERAPY,” filed Mar. 9, 2010, which claims priority to U.S. Provisional Application Ser. No. 61/209,502 entitled “INFECTIOUS DISEASE CELLULAR IMMUNOTHERAPY,” filed Mar. 9, 2009, the disclosure of each is hereby incorporated by reference as if set forth in its entirety herein.

### BACKGROUND

A mammalian immune system uses two general mechanisms to actively protect the body against invading environmental pathogens. One is a non-specific (or innate) inflammatory response. The other is a specific, acquired (or adaptive) immune response. Innate responses are fundamentally the same for each insult or injury while each adaptive response is custom tailored to a specific pathogen. Each adaptive response increases in intensity with each subsequent exposure, which is why they are called specific and adaptive responses.

The immune system recognizes and responds to structural differences between self and non-self proteins expressed by foreign agents including, for example, pathogenic microorganisms. Proteins that the adaptive immune system recognizes as non-self proteins are called “antigens”. Pathogenic microorganisms express large numbers of complex antigens. Adaptive immunity has specific “memory” for antigens such that repeated exposure to the same antigen increases the potency of the adaptive immune response, which increases the level of induced protection against that particular pathogen.

Adaptive immunity is mediated by specialized immune cells called B- and T-lymphocytes. The ability of subpopulations of B- and T-lymphocytes to recognize and respond against antigens expressed by pathogens accounts for the specificity of adaptive immune responses. Additionally, B- and T-lymphocytes are able to replicate themselves upon exposure to antigens. This ability of the B- and T-lymphocytes to replicate following exposure to antigens accounts for an increase in intensity of the adaptive immune responses with repeated exposure to those antigens. Antigen-stimulated B- and T-lymphocytes are also very long-lived, which accounts for an adaptive immunologic memory.

B-lymphocytes produce, secrete, and mediate their functions through the actions of antibodies. B-lymphocyte-dependent immune responses are referred to as “humoral immunity” because antibodies are detected in body fluids (i.e., the humors), such as blood and secretions. Antibodies bind directly to antigens and protect against pathogens in a variety of ways. For example, antibodies may neutralize a toxin produced by the pathogen or may increase the rate of elimination of pathogens by linking the pathogen to cells of the innate immune system, such as macrophages and granulocytes, thereby promoting their being eaten (i.e., phagocytosed) and digested by those innate immune cells. Immunologists call this process “opsonization”. Antibodies have their major protective effects against bacteria.

T-lymphocytes mediate their functions through the activities of effector T-lymphocytes. T-lymphocyte-dependent immune responses are referred to as “cell-mediated immunity”, because cells, e.g., T-lymphocytes and macrophages, mediate effector activities of this arm of the immune system. The local actions of effector T-lymphocytes are amplified through synergistic interactions between effector T-lymphocytes and secondary effector cells, such as macrophages. Effector T-lymphocytes produce molecules called cytokines that activate macrophages to kill pathogens. Cytokines increase macrophages' ability to phagocytose and digest and/or kill pathogens. Cell-mediated immunity plays a major role in resistance to viruses, fungi, parasites, cancers, and bacteria, such as Mycobacterium tuberculosis, that have the ability to live within cells of the innate immune system and sometimes also within other cells in the body.

Protection assays may make it possible to determine if a substance is antigenic or if an acquired immune response has been induced in an individual that has been exposed to an antigen. Most human infectious agents are not pathogens for non-human animals and the general purpose of performing a protection assay is to determine the efficacy of a particular immunization strategy for humans. For ethical reasons, experimental protection experiments may not be used to measure antigen-induced cell-mediated immune responses against pathogens in humans. Randomized, placebo-controlled clinical trials may be performed using populations of individuals at high risk for developing a disease in question to determine an ability of a test vaccination strategy to reduce disease incidence. This strategy is extremely expensive and is a time-consuming method.

There is a myriad of in vitro assays for measuring an increase in serum (i.e., the fluid portion of blood) antibody levels that can be used to measure humoral immunity. It is, however, much more difficult to determine that a cell-mediated immune response has been induced since it is difficult to measure increases in circulating levels of antigen-specific T-lymphocytes. Historically, antibody levels have been used as surrogate measures, but do not directly measure protection and do not measure cell mediated immune responses.

In vivo protection assays have proven to be the most reliable measures of cell-mediated immune responses against pathogens. Thus, an individual would be immunized with the antigen in question and then challenged with the pathogenic agent. This allows one to determine whether protection has been induced. Still, the degree of protection may be difficult to quantify. Protection assays may be appropriate when an antigen in question causes disease and when the studies are being performed in experimental models. Thus, mice would be exposed to one or more viral antigens and then injected later with the live virus. If no disease develops, then the animal is immune and it may be inferred that a protective immune response was induced against that pathogenic agent. Protective assays may also be used to determine the specificity of immune responses.

There are no simple, reliable, quantitative in vitro assays for cell-mediated immunity since cell-mediated immunity is a complex interactive process that involves the coming together of several cell types in different tissues in vivo. The only in vivo assay that fits all of these criteria is the delayed type hypersensitivity (DTH) skin testing assay. The DTH skin testing assay takes advantage of the fact that when antigens are injected into the skin of a previously immunized animal or human, the injected individual will, if immune, develop an acquired cell-mediated immune reaction in the injection site that is characterized by redness and swelling. The size of the reaction can be measured and is a direct reflection of the intensity of the immune response that developed following vaccination. Of course, the DTH reaction is still considered to be a surrogate test for immune protection. Protective cancer immunity has been shown to correlate with DTH responses to cancer antigens in animal models.

The DTH reaction is the major method that has been used so far to measure cell-mediated immune responses against antigens in vivo in humans. DTH responses, like protective immunity, are mediated locally by a combination of activated Thi-lymphocytes and non-cytotoxic, Th1-like CD8⁺ T-lymphocytes.

A variety of medical interventions that augment the body's adaptive immune response(s) to pathogens have been developed. Medical interventions make use of the fact that acquired immune responses can be artificially manipulated. Those medical interventions are classified either as active or passive. Active immunological interventions may include, for example, exposing individuals to a weakened or inactivated pathogen that induces acquired immunity without causing disease and, additionally, protects the individual against later exposure to the same pathogen. The general process of artificially inducing protective immune responses is called immunization or vaccination. Vaccines are used for immunization and are extremely useful for disease prevention. Immunizations have been used to induce protection against a wide variety of environmental pathogens, particularly viruses. Still, the preventative value of immunizations may be limited. Immunization's preventative value may be limited for diseases that do not affect high numbers of individuals in the population because it is difficult to justify the expense of population-wide immunization for a disease that only affects a small number of individuals. Additionally, there are several infectious diseases that have resisted the development of an effective vaccine. In those situations, immunization apparently fails to induce protective responses in a significant proportion of infected individuals. Despite limitations, immunizations have achieved dramatic preventative success that has led to a search for therapeutic applications including, for example, the search for a therapeutic AIDS vaccine. However, vaccines have historically had little effect on disease progression once infection has been initiated.

Adaptive protective immunity can be passively transferred from one genetically identical individual to another, for example, in experimental model systems. Passive transfer experiments provide the methodology for determining whether antibodies, T-lymphocytes, or a combination thereof mediates immunity to a particular pathogen. Passive transfer has been used to establish that T-lymphocytes mediate viral immunity, immunity to obligate intracellular pathogens, and cancer immunity. T-lymphocytes transferred from an immune individual to a non-immune individual provide immune protection for the non-immune individual.

An example of a passive medical immunological intervention would be injecting a snakebite victim with anti-venom (i.e., antibodies specifically directed against the snake's toxic venom) or injecting an individual that has incurred a deep wound, who has never been vaccinated against tetanus toxin, with antibodies directed against the tetanus toxin. Protective immunity to some pathogenic agents can be transferred from one individual to another using T-lymphocytes. The fact that immunity to those pathogens may be transferred between individuals using T-lymphocytes, but not antibodies, has been interpreted to mean that T-lymphocytes mediate immunity to those pathogens.

Passive transfer of immune T-lymphocytes between individuals can be accomplished in genetically identical animal models but is impractical as a medical intervention in humans unless the recipient is severely immune compromised because the genetic differences between individual humans would lead to T-lymphocytes being rapidly rejected by the recipient's immune system. That is, the recipient's immune system recognizes the donor T-lymphocytes as non-self, develops an immune response against them, and rapidly eliminates them from the body. However, passive transfer of T-lymphocytes in the same individual, (i.e., auto transplantation of T-lymphocytes or autologous adoptive transfer of T-lymphocytes) is feasible and safe as a medical intervention. Currently, there are no U.S. Food and Drug Administration (FDA) approved medical interventions that employ T-lymphocyte transfer between individuals, but autotransplantation of bone marrow or peripheral blood containing T-lymphocytes as a source of stem cells following high dose chemotherapy or radiation is routinely performed as a medical intervention. As noted above, autotransplantation of T-lymphocytes from a vaccinated individual to an unvaccinated individual, e.g., between genetically identical rodents, transfers protection, but, as is the case for vaccination itself, fails to transfer significant therapeutic efficacy. That is, transfer of T-lymphocytes from immune individuals to infected individuals fails to terminate established infections.

The specific adaptive immune system naturally terminates infections on a regular basis. An example of such regular termination is the human body's response to an influenza virus infection. The infected individual may become ill but almost invariably will completely recover unless the immune system has somehow been weakened, for example, by age or by immunosuppressive drugs. Nevertheless, extensive research has illustrated that vaccines do not control active disease.

One hypothesis to explain the failure of transferring T-lymphocytes from immunized individuals to eliminate established disease in diseased individuals is that vaccination generates significant numbers of antigen-specific effector T-cell precursors, but few fully activated effector T-cells. That hypothesis was tested by generating effector T-cells from those partially activated precursors in vitro and passively transferring them to individuals with active disease. When that was done, active infections could be terminated. For example, clones of effector T-lymphocytes and enriched populations of cytotoxic T-lymphocytes will terminate active infections.

Another explanation for vaccine failure is that the immune response induced by vaccination is quantitatively deficient, i.e., “too little, too late”. Vaccines are opined to be effective as preventative interventions because, at the time of initial infection, the infected individual is exposed to small numbers of the pathogen and re-exposure of the individual to the pathogen generates a sufficiently high number of effector T-lymphocytes to eliminate the small viral load at the site of entry. It follows logically from this that therapeutic vaccines do not generate a sufficiently high number of effector T-lymphocytes to eliminate the high viral load present during active infection.

Another possible explanation would be that vaccination induces a qualitatively deficient immune response that is effective for prevention because re-exposure to a virus at virus entry sites is a requisite to generate effector T-lymphocytes. In fact, it is well established in the immunologic literature that vaccination only produces increased numbers of partially activated T-lymphocytes, known as primed T-lymphocytes. Few, if any, effector T-lymphocytes can be detected in a vaccinated individual. In order to terminate an active infection, a medical intervention would have to substantially increase the number of fully activated effector T-lymphocytes that are both circulating in the body and able to reach sites of viral spread.

This logic underlies the development of cloning effector T-lymphocytes for use as therapeutic tools in viral disease. It should be noted that the logic that underlies the cloning approach is non-autologous. That is, the general idea is that one could use cytotoxic T-lymphocytes as a pharmaceutical. For example, all cytomegalovirus (CMV) patients would receive an infusion of the same T-lymphocyte clone that was originally generated from a genetically different individual. To make this technically feasible, methods have been developed for increasing the long-term survival of these non-autologous cells in genetically disparate individuals. In other words, the cells are genetically programmed to resist immune destruction.

Generally, studies have found that passive transfer of antigen-specific effector T-lymphocytes into diseased animals would eliminate the disease-causing agent (e.g., cancer cells) and cure the animals. It is important to note that, in some of the experimental models, the T-lymphocytes that eliminated cancer cells had specificity for viral antigens expressed by the cancer cell. In an early study, it was demonstrated that the strategy of passively transferring effector T-lymphocytes that were generated in vitro and that had specificity for a virus that induced leukemia would permanently cure mice dying of murine leukemia. That is, killing virus-infected cancer cells could eliminate cancer cells and the virus. A more recent study using a virally induced cancer demonstrated that effector T-lymphocytes that exhibited specificity for viral antigens eliminated growing cancers and cured the treated animals.

Those general findings led to development of a strategy for treating human cancers, called Cancer Antigen Immunotherapy, as described in U.S. Pat. No. 6,406,699. The Cancer Antigen Immunotherapy combines a cancer vaccination to induce immunity against the patient's cancer with passive transfer of effector T-lymphocytes to eliminate the growing cancer. However, cancers, in contrast to viruses, are slow growing. Also, cancer cells are directly killed by immune cells while few microbes are directly killed by immune cells. Microbes such as viruses are very different from cancer cells in that microbes have evolved many methods for avoiding immune detection once an infection has become established. Thus, for example, microbes can hide in infected cells. Vaccines have not been used to therapeutically treat infectious diseases.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present invention generally related to methods for, among other things, treating various types of infectious diseases in humans using immunotherapy. Specifically, embodiments of the present invention relate to a combination of active immunotherapy (e.g., vaccination) and passive immunotherapy (e.g., infusion of effector T-lymphocytes) for use in treating various types of infectious disease in humans. Patients with an infectious disease may be vaccinated with a vaccine that is designed to induce protection against the infectious disease causing agent. In some embodiments, the vaccine could be combined with an immunologic adjuvant to induce a more powerful immune response against the infectious agent than would be induced by the antigen alone. Infectious agent antigen-primed peripheral blood T-lymphocytes may be removed from the patient. The antigen-primed T-lymphocytes may be stimulated to differentiate into effector T-lymphocytes in vitro. The effector T-lymphocytes may be stimulated to proliferate in vitro, thereby increasing their numbers. The effector T-lymphocytes may be intravenously infused back into the patient.

Additional objects, advantages, and novel features of the invention will be set forth in part in the description which follows, and in part will become apparent to those skilled in the art upon examination of the following, or may be learned by practice of the invention.

### DETAILED DESCRIPTION

The subject matter of embodiments of the present invention is described with specificity herein to meet statutory requirements. However, the description itself is not intended to limit the scope of this patent. Rather, the inventor has contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

Embodiments of the present invention are generally directed to treating various types of infectious diseases in humans using immunotherapy. Utilizing methods described herein, a patient having an infectious disease is vaccinated with a vaccine that is designed to induce protection against the infectious disease-causing agent. The disease may be active or quiescent at the time of vaccination. Or, the individual may simply be infected but the active disease may not yet have occurred as in the case of HIV infection that eventually progresses to AIDS. Infectious agent antigen-primed peripheral blood T-lymphocytes may be removed from the patient and may be stimulated to differentiate into effector T lymphocytes in vitro. The resulting effector T-lymphocytes may be intravenously infused back into the patient.

Accordingly, in one aspect, an embodiment of the present invention is directed to a method for treating an infectious disease in a person. The method includes vaccinating a person having an infectious disease with a vaccine designed to induce an immune response against an infectious agent causing the infectious disease. Primed T-lymphocytes are then removed from the person. The primed T-lymphocytes are stimulated, in vitro, to differentiate into effector T-lymphocytes. The effector T-lymphocytes are stimulated, in vitro, to proliferate. The effector T-lymphocytes are infused back into the person.

In another aspect, an embodiment of the present invention is directed to a method for treating an infectious disease in a person. The method includes administering a first vaccine to a person having an infectious disease. The first vaccine is designed to induce an immune response against an infectious agent causing the infectious disease. Upon administering the first vaccine, an immune response exhibited by the person is identified. Based on a determination that the immune response is below a predetermined immune response threshold, a second vaccine is administered to the person. Primed T-lymphocytes are removed from the person using apheresis. The primed T-lymphocytes are treated, in vitro, with a T-lymphocyte stimulus that stimulates the primed T-lymphocytes to differentiate into effector T-lymphocytes. The effector T-lymphocytes are stimulated, in vitro, with a cytokine that stimulates proliferation of effector T-lymphocytes. The effector T-lymphocytes are infused back into the person.

Having briefly described an overview of embodiments of the present invention, exemplary methods suitable for use in implementing embodiment of the present invention are described below.

Embodiments of the present invention immunize (i.e., vaccinate) patients with antigens from an agent causing the patient's disease. The vaccines contemplated for use in accordance with the present invention include, but are not limited to, bacterial vaccines, fungal vaccines, viral vaccines used for active immunization, and the like. Exemplary vaccines may be found in a list of approved vaccines maintained by the FDA. Suitable vaccines include, but are not limited to, vaccines against the following disease entities or disease-causing organisms: tuberculosis, measles, mumps, rubella, diphtheria, pertussis, Hemophilus influenza, tetanus, hepatitis B, polio, anthrax, plague, encephalitis, meningococcal, meningitis, pneumococcus, typhus, typhoid fever, streptococcus, staphylococcus, neisseria, lyme, cytomegalovirus (CMV), respiratory syncytial virus, Epstein Barr virus, herpes, influenza, parainfluenza, rotavirus, adenovirus, human immunodeficiency virus (HIV), hepatitis A, NonA NonB hepatitis, varicella, rabies, yellow fever, Japanese encephalitis, flavivirus, dengue, toxoplasmosis, cocidiomycosis, schistosomiasis, and malaria.

In embodiments, vaccines that have not been approved for disease prevention by the FDA may also be used including, for example, designer vaccines such as molecular vaccines that have been designed based on an understanding of a genetic makeup of an agent in question and an identity of immunodominant microbial antigens. Such designer vaccines may not generate life-threatening side effects that are sometimes seen with live, attenuated and dead whole virus vaccines that are caused by anaphylactic reactions to components of the vaccine.

Once the person having the infectious disease has been vaccinated with the vaccine designed to induce protection against the infectious disease causing agent, an immune response exhibited by the person may be measured. The immune response may depend on the vaccine administered to the individual, the individual, or a combination thereof. Immune responses may be measured by any known methods including, but not limited to, measuring antibody levels and measuring a DTH reaction. Immune responses may be determined to be weak or strong based on a predetermined immune response threshold. The predetermined immune response threshold may be based on any immune response standards known in the art.

An immune response may depend on the specific vaccine administered. For example, a vaccine for Disease A may be a very strong vaccine that renders an immune response that is determined to be very strong. However, a vaccine for Disease B may render an immune response that is determined to be very weak. The immune response may also depend on attributes of the person including, an age of the person, a status of the person's immune system (i.e., whether the immune system of the person is compromised), or the like.

Based on the immune response exhibited by the person, a determination may be made whether subsequent vaccines are required. An immune response of a predetermined threshold may be desired. Thus, an immune response that is below the predetermined threshold may indicate that a subsequent vaccine is required while an immune response that is equal to or greater than the predetermined threshold may indicate that a single vaccine is sufficient to proceed. Upon determining that the immune response is equal to or greater than the predetermined threshold, no additional vaccines may be necessary. Upon determining that the immune response is less than the predetermined threshold, additional vaccines may be administered to the person until an immune response is observed that is equal to or greater than the predetermined threshold. In embodiments, multiple vaccines may be administered before an immune response is initially measured.

In an embodiment, an immunologic adjuvant may be combined with the vaccine initially or upon determining that the immune response is less than the predetermined threshold. Suitable immunological adjuvants that may be included in a vaccine include several classes of human adjuvant. Those include, but are not limited to, mineral salts, surface active agents, microparticles, bacterial products, cytokines, hormones, unique antigen constructs, polyanions, dendritic cells, or the like. In an embodiment, the immunologic adjuvant is a granulocyte macrophage colony stimulating factor (GM-CSF).

The vaccine may be administered in any injection site(s) appropriate for administering a vaccination and may be administered in a single injection site or multiple injection sites. In embodiments, the injection site(s) is determined such that maximum exposure of the antigen to the highest number of draining lymph nodes is accomplished. For example, there are a large number of draining lymph nodes located in both the groin and axillae areas. Thus, it may be determined that a vaccine should be administered to both the groin and axillae areas to maximize the number of draining lymph nodes that are exposed to the antigen.

Once an immune response is measured that satisfies the predetermined threshold, primed T-lymphocytes may be removed from the vaccinated individual. Vaccination leads to production of primed antigen-specific T-lymphocytes in lymphoid tissue draining the vaccination sites. The primed T-lymphocytes are released from lymphoid tissue into the blood so that they may be carried to the sites of the antigen exposure, i.e., sites of active disease, where, if conditions were optimal, they would be stimulated to differentiate into effector T-lymphocytes that would kill microbe infected cells and terminate the infection. Since primed T-lymphocytes would be rapidly released into the blood, peripheral blood may provide the richest source of antigen specific effector T-lymphocyte precursors. Primed T-lymphocytes may be obtained from lymph nodes draining vaccination sites that would be removed surgically or from other lymphoid tissue. In an embodiment, apheresis may be used to obtain blood that contains high numbers of primed T-lymphocytes. Any other method known may be used to obtain peripheral blood T-lymphocytes. In embodiments, apheresis may be performed within two weeks following the last exposure to the vaccine.

Once the primed T-lymphocytes are removed from the person, activation and proliferation of the primed T-lymphocytes may be induced during in vitro cell culture as a result of a cooperative interaction between adherent monocytes and dendritic cells and non-adherent T-lymphocytes. Blood mononuclear cells may be cultured in plastic tissue culture flasks that allow cell attachment in a cell culture medium containing the patient's serum. In embodiments, autologous serum may be used. Additional serum sources may be substituted for the autologous serum or the cells may be cultured in a serum-free medium.

The peripheral blood T-lymphocytes removed from the person may be nonspecifically stimulated in a culture with antibodies directed against CD3, which recognize a component in the T-lymphocyte antigen receptor complex. Anti-CD3 stimulates primed antigen-specific T-lymphocytes to differentiate into antigen-specific effector T-lymphocytes. Other non-specific T-lymphocyte stimuli including, but not limited to, staphylococcus enterotoxin or bryostatin-1, may be substituted for the anti-CD3. While the stimulus may not be able to bind to the antigen receptor or to antigen receptor-associated proteins, the stimulus is capable of stimulating primed T-lymphocytes to differentiate into effector T-lymphocytes that maintain their antigen specificity and effector T-lymphocyte activity. The blood T-lymphocytes removed from the person may be exposed to anti-CD3 for a predetermined period of time. In an embodiment, the blood T-lymphocytes may be exposed to anti-CD3 for 24-48 hours. A concentration of anti-CD3 for stimulating activation of T lymphocytes may be between 0.01 and 100 nanograms/milliliter.

In embodiments, interleukin-2 (IL-2), which binds to a T-lymphocyte IL-2 receptor and stimulates T-lymphocytes to proliferate, may be added to the cultures after T-lymphocyte activation (i.e., differentiation) has been accomplished. Any other cytokine capable of stimulating proliferation of T-lymphocytes, such as IL-15, may be substituted for IL-2. In addition, other cytokines could be added to the mixture that would increase the T-lymphocyte yield in the culture by, for example, promoting T-lymphocyte viability. In an embodiment, a concentration of IL-2 for stimulating proliferation of activated T-lymphocytes may be between 1.0 and 1000 IU/milliliter.

In additional embodiments, the T-lymphocytes may be specifically stimulated with antigen or molecular constructs of antigens, preferably those that are immunodominant, from the infectious agents alone or in combination with non-specific stimuli, such as anti-CD3, and then stimulated with IL-2.

Once the stimulated cells have been harvested from culture, the cells may be infused intravenously into the patient from whom they were originally obtained. In an embodiment, the patient may be infused with 10¹⁰ to 10¹² lymphocytes over a period of 1-6 hours. However, the number of mononuclear cells administered is dependent upon the number of cells generated during the activation and proliferation steps. In embodiments, over 10¹² activated autologous lymphocytes have been safely infused into patients.

Once the stimulated cells have been infused back into the patient's bloodstream, the patient may receive an administration of subcutaneous or intravenous IL-2 in order to stimulate continued proliferation of the activated T-lymphocytes after they have been delivered back into the person's body. Any other cytokine capable of stimulating proliferation of T-lymphocytes, such as IL-15, may be used.

This strategy increases the number of effector T-lymphocytes circulating in the infected individual's body using autologous polyclonal T-lymphocyte populations. This strategy is based on the fact that when primed T-lymphocytes are removed from vaccinated animals, they exhibit little effector T-lymphocyte activity. But, re-exposing the antigen-primed T-lymphocytes to the antigen in vitro produces effector T-lymphocytes. Thus, one could deliver the activated effector T-lymphocytes to diseased individuals and tip the host/invader balance to terminate infection.

By way of example only, a person infected with Hepatitis B may be vaccinated in four separate intradermal sites (e.g., left and right axillae and left and right groin) with a Hepatitis B vaccine combined with GM-CSF as an immunologic adjuvant. The multiple injections may be determined to maximize the exposure of the antigen to the highest number of draining lymph nodes. In an embodiment, the GM-CSF may be added to the vaccine after an initial administration of the vaccine to the person. Alternatively, the GM-CSF may be administered to the person after the vaccine. Other adjuvants or vaccine formulations may also be effective.

The person infected with Hepatitis B may be injected with GM-CSF daily for at least three days at the original vaccination sites to maintain heightened local levels of adjuvant. Multiple injections of adjuvants may depend on the immune response exhibited by the individual, the vaccine/adjuvant combination administered, or the like. Since the Hepatitis B vaccine/adjuvant combination exerts minimal toxicity, multiple vaccinations may be safely performed to improve the immune response exhibited by the person. The vaccination results in an increased number of circulating antigen-primed T-lymphocytes in the patient's body and, thus, increases the cell-mediated immune response against the infectious disease.

From the foregoing, it will be seen that this invention is one well adapted to attain all the ends and objects hereinabove set forth together with other advantages which are obvious and which are inherent to the structure.

It will be understood that certain features and subcombinations are of utility and may be employed without reference to other features and subcombinations. This is contemplated by and is within the scope of the claims.

Since many possible embodiments may be made of the invention without departing from the scope thereof, it is to be understood that all matter herein set forth is to be interpreted as illustrative and not in a limiting sense.

What is claimed is:

1. A method for treating an infectious disease in a person comprising:
vaccinating a person having an infectious disease with a vaccine designed to induce an immune response against an infectious agent causing the infectious disease;
removing primed T-lymphocytes from the person;
stimulating the primed T-lymphocytes to differentiate into effector T-lymphocytes in vitro;
stimulating the effector T-lymphocytes to proliferate in vitro; and
infusing the effector T-lymphocytes back into the person.

2. The method of claim 1, further comprising:
upon infusing the effector T-lymphocytes to the person, infusing the person with interleukin-2.

3. The method of claim 1, wherein the vaccine includes an immunologic adjuvant.

4. The method of claim 3, wherein the immunologic adjuvant is a granulocyte macrophage colony stimulating factor.

5. The method of claim 1, wherein the primed T-lymphocytes are removed from the person using apheresis.

6. The method of claim 1, wherein the T-lymphocytes are stimulated to differentiate using anti-CD3.

7. The method of claim 1, wherein the infectious disease is HIV.

8. The method of claim 1, wherein the person is vaccinated at a plurality of injection sites.

9. The method of claim 1, wherein the person is vaccinated a plurality of times.

10. The method of claim 1, wherein the person is vaccinated at the time of initial diagnosis.

11. The method of claim 1, wherein the person is vaccinated with subpopulations of activated T-lymphocytes.

12. A method for treating an infectious disease in a person comprising:
administering a first vaccine to a person having an infectious disease, wherein the first vaccine is designed to induce an immune response against an infectious agent causing the infectious disease;
upon administering the first vaccine, identifying an immune response exhibited by the person;
based on a determination that the immune response is below a predetermined immune response threshold, administering a second vaccine to the person;
removing primed T-lymphocytes from the person, wherein the primed T-lymphocytes are removed using apheresis;
treating the primed T-lymphocytes, in vitro, with a T-lymphocyte stimulus that stimulates the primed T-lymphocytes to differentiate into effector T-lymphocytes;
treating the effector T-lymphocytes, in vitro, with a cytokine that stimulates proliferation of effector T-lymphocytes; and
infusing the effector T-lymphocytes back into the person.

13. The method of claim 12, further comprising:
combining the first vaccine with an immunologic agent to yield the second vaccine.

14. The method of claim 13, wherein the immunologic adjuvant is a granulocyte macrophage colony stimulating factor.

15. The method of claim 12, wherein the first vaccine and the second vaccine are the same.

16. The method of claim 12, wherein the cytokine that stimulates proliferation of the effector T-lymphocytes is interleukin-2.

17. The method of claim 12, wherein the T-lymphocyte stimulus that stimulates the primed T-lymphocytes to differentiate into effector T-lymphocytes is anti-CD3.

18. The method of claim 17, wherein a concentration of anti-CD3 is between 0.01 and 100 nanograms/milliliter.

19. The method of claim 12, wherein the primed T-lymphocytes are removed from the person using apheresis.

20. The method of claim 12, further comprising:
upon infusing the person with the effector T-lymphocytes, infusing the person with interleukin-2.
